(19) United States
(12) Patent Application Publication
(10) Pub. No.: US 2016/0097067 A1
(43) Pub. Date: Apr. 7, 2016

(54) METHODS AND SYSTEMS FOR CARBON DIOXIDE SEQUESTRATION

(71) Applicant: Sigma-Aldrich Co., LLC, Saint Louis, MO (US)

(72) Inventors: Ettigounder Ponnusamy, Saint Louis, MO (US); Venkatesalu Bakthavachalam, Natick, MA (US); Steven Walton, Saint Louis, MO (US)

(21) Appl. No.: 14/873,999

(22) Filed: Oct. 2, 2015

Related U.S. Application Data
(60) Provisional application No. 62/058,808, filed on Oct. 2, 2014.

(51) Int. Cl. C12P 9/00

(52) U.S. Cl. CPC C12P 9/00

(57) ABSTRACT

Methods and systems for sequestering carbon dioxide and, in particular, methods and systems for sequestering carbon dioxide by reacting carbon dioxide with an organic substrate, a carboxylase and a salt. Processes for producing 3-phosphoglycerate are also provided. In addition, uses for the 3-phosphoglycerate produced by the methods and systems are provided.

### CROSS-REFERENCE TO RELATED APPLICATIONS

This application claims the benefit of U.S. Provisional Application No. 62/058,808, filed on Oct. 2, 2014. The entire disclosure of the above application is incorporated herein by reference.

### FIELD

The present disclosure relates to carbon dioxide sequestration and, in particular, enzyme-based methods and systems for the preparation of 3-phosphoglycerate from carbon dioxide.

### BACKGROUND

Global climate change is a well-established and an ever increasing problem. A significant contributor to climate change is the unregulated release of carbon dioxide (CO₂) into the atmosphere from the burning of fossil fuels in large point sources, such as electricity generating power plants. To combat climate change, many countries are enacting strict carbon dioxide emission limits. These regulations, among other things, will require large point source emitters of carbon dioxide to reduce emissions by capturing and sequestering the CO₂.

In general, carbon capture and sequestration (CCS) as applied to CO₂ involves several steps including: (a) capturing CO₂ from a source such as, for example, the output of an energy production process; (b) conveying the CO₂ to a treatment site; and (c) treating the CO₂ to prepare it for storage or disposal. To be commercially feasible, any CCS process should be fast, efficient, economical and minimally impactful on the environment. Although there are currently several options for CCS, the known technologies suffer from a variety of inefficiencies. Some require expensive transportation of materials. Others require permanent storage of sequestered CO₂. And still others are difficult or expensive to implement at existing and/or new facilities.

Accordingly, there is a continued need for improved methods and systems for carbon dioxide sequestration. Such methods and systems should be suitable for handling large scale amounts of carbon dioxide. Also, the methods and systems should be commercially viable. In addition, the methods and systems should be cost effective. It would also be beneficial for the methods and systems to be easy to retrofit to old power plants and be suitable for use with future power plants.

### SUMMARY

In one embodiment, the present disclosure provides methods of sequestering carbon dioxide comprising a reacting step, wherein carbon dioxide is reacted with an organic substrate, a carboxylase, and a salt under conditions suitable to form a large scale amount of 3-phosphoglycerate. In one particular embodiment, the methods can form at least 400,000 metric tons/year of 3-phosphoglycerate. In another particular embodiment, the methods can form at least 600,000 metric tons/year of 3-phosphoglycerate. In yet another particular embodiment, the methods can form at least 800,000 metric tons/year of 3-phosphoglycerate. In a further particular embodiment, the methods can form at least 1,000,000 metric tons/year of 3-phosphoglycerate.

In one embodiment, the organic substrate is a D-ribulose 1,5-bisphosphate sodium salt hydrate.

In another embodiment, the carboxylase is a D-ribulose 1,5-diphosphate carboxylase.

In yet another embodiment, the salt is magnesium chloride.

In a further embodiment, the reacting step is performed in a solution. In one embodiment, the solution comprises a buffer. In another embodiment, the solution further comprises an optional anti-foaming agent.

In still a further embodiment, the methods comprise separating the carbon dioxide from a mixture of gases.

In another embodiment, the methods comprise obtaining the carboxylase from spinach.

In yet another embodiment, the methods comprise forming the ribulose 1,5-diphosphate carboxylase by a batch process. In an alternate embodiment, the methods comprise forming the ribulose 1,5-diphosphate carboxylase by a flow process.

In still a further embodiment, the reacting step comprises reacting carbon dioxide with the organic substrate, the carboxylase, and the salt at a temperature of about 15 to about 50° C.

In another embodiment, the reacting step comprises reacting carbon dioxide with the organic substrate, the carboxylase, and the salt for about 1 to about 6 hours.

In yet another embodiment, the reacting step comprises reacting carbon dioxide with the organic substrate, the carboxylase, and the salt at a pH of about 6 to about 8.

In a further embodiment, the methods further comprise isolating the 3-phosphoglycerate. In one embodiment, the isolating the 3-phosphoglycerate comprises separating the 3-phosphoglycerate from a solvent. In another embodiment, the isolating the 3-phosphoglycerate further comprises optionally recycling the solvent. In yet another embodiment, the solvent is water. In a further embodiment, the isolating the 3-phosphoglycerate comprises precipitating the 3-phosphoglycerate. In still a further embodiment, the isolating the 3-phosphoglycerate comprises drying the 3-phosphoglycerate. In one particular embodiment, the drying the 3-phosphoglycerate comprises spray drying the 3-phosphoglycerate.

In another embodiment, the present disclosure provides processes for forming a 3-phosphoglycerate comprising a reacting step, wherein carbon dioxide is reacted with an organic substrate, a carboxylase, and a salt under conditions suitable to form a large scale amount of 3-phosphoglycerate. In one particular embodiment, the processes can form at least 400,000 metric tons/year of 3-phosphoglycerate. In another particular embodiment, the processes can form at least 600,000 metric tons/year of 3-phosphoglycerate. In yet another particular embodiment, the processes can form at least 800,000 metric tons/year of 3-phosphoglycerate. In a further particular embodiment, the processes can form at least 1,000,000 metric tons/year of 3-phosphoglycerate.

In yet another embodiment, the present disclosure provides systems for sequestering carbon dioxide comprising a main reactor. The main reactor comprises a carbon dioxide inlet for introducing carbon dioxide into the main reactor; and a 3-phosphoglycerate outlet for removing 3-phosphoglycerate from the main reactor. In addition, the main reactor defines a reaction zone for reacting the carbon dioxide with an organic substrate, a carboxylase and a salt. In one particular embodiment, the reaction zone is at least 100,000 L.

In one embodiment, the systems comprise a carboxylase inlet for introducing a carboxylase into the main reactor.

In another embodiment, the systems comprise a flow reactor operatively connected to the carboxylase inlet for continuously flowing the carboxylase into the main reactor.

In yet another embodiment, the systems comprise an anti-foaming agent inlet for introducing an anti-foaming agent into the main reactor.

In a further embodiment, the systems comprise a precipitation reactor operatively connected to the 3-phosphoglycerate outlet for receiving a solution comprising the 3-phosphoglycerate and a solvent from the main reactor, wherein the precipitation reactor defines a precipitation zone for reacting the solution with a precipitation agent to form a precipitate comprising the 3-phosphoglycerate. In one embodiment, the precipitation reactor comprises a precipitation agent inlet for introducing the precipitation agent into the precipitation reactor. In another embodiment, a centrifuge is operatively connected to the precipitation reactor for receiving the 3-phosphoglycerate and the solvent and for separating the 3-phosphoglycerate from the solvent. In one embodiment, a recycle stream is operatively connected to the centrifuge for receiving the solvent and operatively connected to the main reactor for supplying the solvent to the main reactor. In another embodiment, a dryer is operatively connected to the centrifuge for receiving and drying the 3-phosphoglycerate.

### DETAILED DESCRIPTION

### I. Definitions

As used herein, the term “sequestration” is used to refer generally to techniques or practices whose partial or whole effect is to remove CO₂ from point emissions sources and, preferably, to store that CO₂ in some form so as to prevent its return to the atmosphere.

The term, “a large scale amount,” refers to an amount suitable for industrial application. In an embodiment, a large scale amount is at least 400,000 metric tons/year. In other embodiments, a large scale amount is at least 600,000 metric tons/year, at least 800,000 metric tons/year, or at least 1,000,000 metric tons/year.

The words “comprise”, “comprises”, and “comprising” are to be interpreted inclusively rather than exclusively.

### II. Methods of Sequestering Carbon Dioxide

In a first aspect of the present disclosure, methods of sequestering carbon dioxide are provided. According to the methods, carbon dioxide is obtained from a suitable source. The carbon dioxide is then reacted with an organic substrate, a carboxylase and a salt under conditions suitable to form 3-phosphoglycerate (also known as (2R)-2-hydroxy-3-phosphonooxypropanoic acid; CAS Number 820-11-1). The 3-phosphoglycerate is then optionally isolated for further use. The reaction of carbon dioxide with the organic substrate, the carboxylase and the salt can be performed under conditions that form a large scale amount of 3-phosphoglycerate. In particular, the methods can form at least 400,000 metric tons/year, at least 600,000 metric tons/year, at least 800,000 metric tons/year, or at least 1,000,000 metric tons/year of 3-phosphoglycerate.

Broadly, the carbon dioxide can be obtained from any one of a variety of sources of either purified carbon dioxide or mixtures of gases comprising carbon dioxide. More specifically, the carbon dioxide can be obtained from flue gases, waste streams, power plant emissions, and combinations thereof. In particular, the carbon dioxide can be obtained by post-combustion capture that separates CO₂ from flue gases produced by combustion of a primary fuel (e.g., coal, natural gas, oil or biomass) in air; pre-combustion capture wherein a primary fuel is processed to produce a separate stream of CO₂; oxyfuel combustion that uses oxygen instead of air for combustion, producing a flue gas that is mainly H₂O and CO₂; and combinations thereof. The flue gases and/or CO₂ streams can be used untreated. Alternatively, the flue gases and/or CO₂ streams can be treated before being reacted with the organic substrate, the carboxylase and the salt. For example, the flue gases and/or CO₂ streams can be treated to increase the concentration of carbon dioxide and/or to remove other unwanted materials (such as particulates, water, etc.).

Any of a variety of known organic substrates can be used. For example, the organic substrate can be a ribulose 1,5-bisphosphate (also known as ribulose 1,5-diphosphate). The ribulose 1,5-bisphosphate can be a D-ribulose 1,5-bisphosphate. Alternatively, the ribulose 1,5-bisphosphate can be an L-ribulose 1,5-bisphosphate. Further, the ribulose 1,5-bisphosphate can be a mixture of D- and L-ribulose 1,5-bisphosphates. In addition, the ribulose 1,5-bisphosphate can be a ribulose 1,5-bisphosphate hydrate. Further, the ribulose 1,5-bisphosphate can be a ribulose 1,5-bisphosphate salt. The ribulose 1,5-bisphosphate salt can be a sodium salt. Alternatively, the ribulose 1,5-bisphosphate salt can be a potassium salt. In particular, the ribulose 1,5-bisphosphate can be a D-ribulose 1,5-bisphosphate sodium salt hydrate.

With respect to the carboxylase, any of a variety of carboxylases can be used provided that the carboxylase is stable at the relevant temperature. For example, the carboxylase can be a ribulose 1,5-diphosphate carboxylase/oxygenase (RUBISCO; also known as ribulose 1,5-diphosphate carboxylase/oxygenase). The ribulose 1,5-diphosphate carboxylase can be a D-ribulose 1,5-diphosphate carboxylase. Alternatively, the ribulose 1,5-diphosphate carboxylase can be an L-ribulose 1,5-diphosphate carboxylase. In addition, the carboxylase can be a naturally occurring RUBISCO. Alternatively, the carboxylase can be a modified RUBISCO (see Chatterjee et al., International Research Journal of Plant Science, 2011, 2(2), 022-024).

The carboxylase can be obtained from a variety of sources. For example, the carboxylase can be obtained from animal sources, plant sources, microorganisms (e.g., bacteria), algae and/or recombinant sources. In particular embodiments, when the carboxylase is D-ribulose 1,5-diphosphate carboxylase, the carboxylase can be isolated from a plant (e.g., spinach, see U.S. Pat. No. 6,245,541 to Robert Houtz; and/or tobacco, see Lin et al., Nature, 2014, 513, 547-558). Alternatively, the D-ribulose 1,5-diphosphate carboxylase can be isolated from a cyanobacteria. In addition, the D-ribulose 1,5-diphosphate carboxylase can be isolated from C4 plants or rhodophytes in C3 plant hosts (see U.S. Patent Publication No. 2009/0172842 (Caspar et al.)). Further, the D-ribulose 1,5-diphosphate carboxylase can be isolated from Escherichia coli K-12 (see Kleman et al., Applied and Environmental Microbiology, 1996, 62:9, 3502-3507).

In addition, the carboxylase can be produced using a batch process. Alternatively, the carboxylase can be produced using a flow process.

Any of a variety of salts can be used. In particular, the salt can be magnesium chloride (MgCl₂). Alternatively, the salt can be potassium chloride (KCl).

The reacting carbon dioxide with the organic substrate, the carboxylase and the salt can be performed in a solution. In particular embodiment, the reacting carbon dioxide with the organic substrate, the carboxylase and the salt is performed in an aqueous solution (i.e., a solution wherein the solvent comprises water).

The solution optionally comprises a buffer. In particular, the buffer can be an organic buffer. Any of a variety of organic buffers having a neutral pH (i.e., a pH of 6.5-7.5) can be used. In particular, the buffer can be 2-amino-2-(hydroxymethyl)-1,3-propanediol (e.g., Trizma® base solution, available from Sigma-Aldrich, St. Louis, Mo., USA). Alternatively, the buffer can be 4-(2-hydroxyethyl)-1-piperazineethanesulfonic acid (HEPES, available from Sigma-Aldrich, St. Louis, Mo., USA). Further, although it would be understood that the concentration of buffer can be varied depending on the buffer being used and the reaction conditions, the concentration of buffer can be between 0.5 and 5M. In a particular embodiment, the concentration of buffer is between 0.8 and 1.2 M. In another particular embodiment, the concentration of buffer is about 1M.

In addition, the solution optionally comprises an anti-foaming agent. The anti-foaming agent serves to prevent the solution from foaming upon stifling, thereby allowing the flow of CO₂ through the solution from being increased. Any of a variety of known anti-foaming agents can be used. For example, the anti-foaming agent can be any of a variety of known anti-foaming agents that function at neutral pH. In particular, the anti-foaming agent can be Antifoam B Emulsion (Sigma-Aldrich, St. Louis, Mo., USA). It will be understood that the concentration of anti-foaming agent will vary depending on a variety of factors; however, the anti-foaming agent will typically be present in a concentration sufficient to allow the solution to be stirred without causing the solution to overflow due to foaming.

The specific reaction conditions employed will depend on many factors, including, but not being limited to, the organic substrate, the carboxylase, the salt and the buffer, if used. Accordingly, the reaction can be performed at under a variety of conditions. However, in particular, the reaction can be conducted at a temperature of between about 15 and about 50° C., between about 25 and about 40° C., or between about 30 and about 35° C. In addition, the reaction can be allowed to progress for between about 1 and about 24 hours, between about 1 and about 6 hours, or between about 1 and about 2 hours. Further, the reaction can be conducted at a pH between about 6 and about 8, or between about 6.5 and about 7.5.

The methods of the present disclosure optionally comprise isolating the 3-phosphoglycerate. The 3-phosphoglycerate can be isolated using any of a variety of methods. In particular, the 3-phosphoglycerate can be isolated by separating the 3-phosphoglycerate from the solvent. In one embodiment, the 3-phosphoglycerate is precipitated from the solvent. Precipitation of the 3-phosphoglycertae can be accomplished using a precipitation agent. It will be understood that the particular precipitation agent used will depend on many factors, including (but not being limited to) the organic substrate, the carboxylase, the salt and the solvent. However, suitable precipitation agents include barium salts, calcium salts and the like.

In addition, the isolating the 3-phosphoglycerate optionally comprises recycling the solvent. The recycled solvent can be obtained when the 3-phosphoglycerate is separated from the solvent. In particular, the recycled solvent can be obtained when the 3-phosphoglycerate is precipitated from the solvent. Further, the recycled solvent can be used for any of a variety of purposes. In particular, however, the recycled solvent can be used when the carbon dioxide is reacted with the organic substrate, the carboxylase and the salt.

The isolating the 3-phosphoglycerate also optionally comprises drying the 3-phosphoglycerate. The 3-phosphoglycerate can be dried using any of a variety of techniques. For example, the 3-phosphoglycerate can be air dried. Alternatively, the 3-phosphoglycerate can be dried in an oven. As another alternative, the 3-phosphoglycerate can be spray dried.

The 3-phosphoglycerate can be isolated for any of a variety of reasons. For example, the 3-phosphoglycerate can be used as an industrial raw material. In addition, the 3-phosphoglycerate can be used as a fertilizer. The 3-phosphoglycerate can also be used to react with ATP to generate ribulose 1,5-biphosphate (see, for example, U.S. Pat. No. 6,258,335 (Bhattacharya)).

### III. Processes for Forming a 3-Phosphoglycerate

In another aspect of the present disclosure, processes for forming 3-phosphoglycerate is provided. The processes comprise reacting carbon dioxide with an organic substrate, a carboxylase, and a salt under conditions suitable to form a large scale amount of the 3-phosphoglycerate. In particular, the processes can form at least 400,000 metric tons/year, at least 600,000 metric tons/year, at least 800,000 metric tons/year, or at least 1,000,000 metric tons/year of 3-phosphoglycerate.

Broadly, the carbon dioxide can be obtained from any of a variety of sources of either purified carbon dioxide or mixtures of gases comprising carbon dioxide. More specifically, the carbon dioxide can be obtained from flue gases, waste streams, power plant emissions, and combinations thereof. In particular, the carbon dioxide can be obtained by post-combustion capture that separates CO₂ from flue gases produced by combustion of a primary fuel (e.g., coal, natural gas, oil or biomass) in air; pre-combustion capture, wherein a primary fuel is processed to produce a separate stream of CO₂; oxyfuel combustion that uses oxygen instead of air for combustion, producing a flue gas that is mainly H₂O and CO₂; and combinations thereof. The flue gases and/or CO₂ streams can be used untreated. Alternatively, the flue gases and/or CO₂ streams can be treated before being reacted with the organic substrate, the carboxylase and the salt. For example, the flue gases and/or CO₂ streams can be treated to increase the concentration of carbon dioxide and/or to remove other unwanted materials (such as particulates, water, etc.).

Any of a variety of known organic substrates can be used. For example, the organic substrate can be a ribulose 1,5-bisphosphate (also known as ribulose 1,5-diphosphate). The ribulose 1,5-bisphosphate can be a D-ribulose 1,5-bisphosphate. Alternatively, the ribulose 1,5-bisphosphate can be an L-ribulose 1,5-bisphosphate. Further, the ribulose 1,5-bisphosphate can be a mixture of D- and L-ribulose 1,5-bisphosphates. In addition, the ribulose 1,5-bisphosphate can be a ribulose 1,5-bisphosphate hydrate. Further, the ribulose 1,5-bisphosphate can be a ribulose 1,5-bisphosphate salt. The ribulose 1,5-bisphosphate salt can be a sodium salt. Alternatively, the ribulose 1,5-bisphosphate salt can be a potassium salt. In particular, the ribulose 1,5-bisphosphate can be a D-ribulose 1,5-bisphosphate sodium salt hydrate.

With respect to the carboxylase, any of a variety of carboxylases can be used provided that the carboxylase is stable at the relevant temperature. For example, the carboxylase can be a ribulose 1,5-diphosphate carboxylase/oxygenase (RUBISCO; also known as ribulose 1,5-diphosphate carboxylase/oxygenase). The ribulose 1,5-diphosphate carboxylase can be a D-ribulose 1,5-diphosphate carboxylase. Alternatively, the ribulose 1,5-diphosphate carboxylase can be an L-ribulose 1,5-diphosphate carboxylase. In addition, the carboxylase can be a naturally occurring RUBISCO. Alternatively, the carboxylase can be a modified RUBISCO (see Chatterjee et al., International Research Journal of Plant Science, 2011, 2(2), 022-024).

The carboxylase can be obtained from a variety of sources. For example, the carboxylase can be obtained from animal sources, plant sources, microorganisms (e.g., bacteria), algae and/or recombinant sources. In particular embodiments, when the carboxylase is D-ribulose 1,5-diphosphate carboxylase, the carboxylase can be isolated from a plant (e.g., spinach, see U.S. Pat. No. 6,245,541 to Robert Houtz; and/or tobacco, see Lin et al., Nature, 2014, 513, 547-558). Alternatively, the D-ribulose 1,5-diphosphate carboxylase can be isolated from a cyanobacteria. In addition, the D-ribulose 1,5-diphosphate carboxylase can be isolated from C4 plants or rhodophytes in C3 plant hosts (see U.S. Patent Publication No. 2009/0172842 (Caspar et al.)). Further, the D-ribulose 1,5-diphosphate carboxylase can be isolated from Escherichia coli K-12 (see Kleman et al., Applied and Environmental Microbiology, 1996, 62:9, 3502-3507).

In addition, the carboxylase can be produced using a batch process. Alternatively, the carboxylase can be produced using a flow process.

Any of a variety of salts can be used. In particular, the salt can be magnesium chloride (MgCl₂). Alternatively, the salt can be potassium chloride (KCl).

The step of reacting carbon dioxide with the organic substrate, the carboxylase and the salt is preferably performed in solution. In particular, the step of reacting carbon dioxide with the organic substrate, the carboxylase and the salt is preferably performed in an aqueous solution (i.e., a solution wherein the solvent comprises water).

The solution optionally comprises a buffer. In particular, the buffer can be an organic buffer. Any of a variety of organic buffers having a neutral pH (i.e., a pH of about 6.5 to about 7.5) can be used. In particular, the buffer can be 2-amino-2-(hydroxymethyl)-1,3-propanediol (e.g., Trizma® base solution available from Sigma-Aldrich, St. Louis, Mo., USA). Alternatively, the buffer can be 4-(2-hydroxyethyl)-1-piperazineethanesulfonic acid (HEPES, available from Sigma-Aldrich, St. Louis, Mo., USA). Further, although it would be understood that the concentration of buffer can be varied depending on the buffer being used and the reaction conditions, the concentration of buffer is preferably between about 0.5 and about 5M. In a particular embodiment, the concentration of buffer is between about 0.8 and about 1.2 M. In a particular embodiment, the concentration of buffer is about 1M.

In addition, the solution optionally comprises an anti-foaming agent. The anti-foaming agent serves to prevent the solution from foaming upon stifling, thereby allowing the flow of CO₂ through the solution from being increased. Any of a variety of known anti-foaming agents can be used. For example, the anti-foaming agent can be any of a variety of known anti-foaming agents that function at neutral pH. In particular, the anti-foaming agent can be Antifoam B Emulsion (Sigma-Aldrich, St. Louis, Mo., USA). It will be understood that the concentration of anti-foaming agent will vary depending on a variety of factors; however, the antifoaming agent will typically be present in a concentration sufficient to allow the solution to be stirred without causing the solution to overflow.

The specific reaction conditions employed will depend on many factors, including, but not being limited to, the organic substrate, the carboxylase, the salt and the buffer, if used. Accordingly, the reaction can be performed at under a variety of conditions. However, in particular, the reaction can be conducted at a temperature of between about 15 and about 50° C., between about 25 and about 40° C., or between about 30 and about 35° C. In addition, the reaction can be allowed to progress for between about 1 and about 24 hours, between about 1 and about 6 hours, or between about 1 and about 2 hours. Further, the reaction can be conducted at a pH between about 6 and about 8, or between about 6.5 and about 7.5.

The methods of the present disclosure optionally comprise isolating the 3-phosphoglycerate. The 3-phosphoglycerate can be isolated using any of a variety of methods. In particular, the 3-phosphoglycerate can be isolated by separating the 3-phosphoglycerate from the solvent. In one embodiment, the 3-phosphoglycerate is precipitated from the solvent. Precipitation of the 3-phosphoglycertae can be accomplished using a precipitation agent. It will be understood that the particular precipitation agent used will depend on many factors, including (but not being limited to) the organic substrate, the carboxylase, the salt and the solvent. However, suitable precipitation agents include barium salts, calcium salts and the like.

In addition, the isolating the 3-phosphoglycerate optionally comprises recycling the solvent. The recycled solvent can be obtained when the 3-phosphoglycerate is separated from the solvent. In particular, the recycled solvent can be obtained when the 3-phosphoglycerate is precipitated from the solvent. Further, the recycled solvent can be used for any of a variety of purposes. In particular, however, the recycled solvent can be used when the carbon dioxide is reacted with the organic substrate, the carboxylase and the salt.

The isolating the 3-phosphoglycerate also optionally comprises drying the 3-phosphoglycerate. The 3-phosphoglycerate can be dried using any of a variety of techniques. For example, the 3-phosphoglycerate can be air dried. Alternatively, the 3-phosphoglycerate can be dried in an oven. As another alternative, the 3-phosphoglycerate can be spray dried.

The 3-phosphoglycerate can be isolated for any of a variety of reasons. For example, the 3-phosphoglycerate can be used as an industrial raw material. In addition, the 3-phosphoglycerate can be used as a fertilizer. The 3-phosphoglycerate can also be used to react with ATP to generate ribulose 1,5-biphosphate (see, for example, U.S. Pat. No. 6,258,335 (Bhattacharya)).

### III. Systems for Sequestering Carbon Dioxide

In yet another aspect of the present disclosure, systems for sequestering carbon dioxide are provided. The systems comprise a main reactor comprising a carbon dioxide inlet and a 3-phosphoglycerate outlet. The main reactor defines a reaction zone. The reaction zone holds the organic substrate, the carboxylase and the salt for reaction with the carbon dioxide. In particular, the reaction zone of the main reactor can be sized on a scale suitable for producing large scale amounts of 3-phosphoglycerate. For example, the reaction zone of the main reactor can be at least 100,000 L, at least 150,000 L, or at least 200,000 L.

When the ribulose 1,5-diphosphate carboxylase is to be formed by a batch process, the carboxylase can be introduced directly into the reaction zone of the main reactor.

Alternatively, a carboxylase source can be introduced into the main reactor so that the carboxylase can be formed in situ within the main reactor. However, when the ribulose 1,5-diphosphate carboxylase is to be formed by a flow process, the carboxylase can be produced outside of the main reactor and then introduced into the reaction zone. Accordingly, the main reactor may optionally comprise a carboxylase inlet to facilitate introduction of the carboxylase or carboxylase source into the main reactor. In addition, the main reactor may optionally comprise an anti-foaming agent inlet for introducing an anti-foaming agent into the main reactor.

The carbon dioxide inlet is used to facilitate introduction of carbon dioxide into the reaction zone of the main reactor. Accordingly, the carbon dioxide inlet can be operatively connected to a carbon dioxide source. In particular, the carbon dioxide source can be a waste stream. Alternatively, the carbon dioxide source can be a flue gas from, for example, a power plant (i.e., a coal burning power plant). A regulator or flow meter is optionally provided for adjusting the flow rate of CO₂.

The 3-phosphoglycerate outlet is used to remove 3-phosphoglycerate from the main reactor. In particular, the 3-phosphoglycerate can be removed from the main reactor as a solution comprising the 3-phosphoglycerate and a solvent. In particular, the solvent can be water.

The systems of the present disclosure may also optionally comprise a precipitation reactor. The precipitation reactor is operatively connected to the 3-phosphoglycerate outlet of the main reactor for receiving the solution comprising the 3-phosphoglycerate and the solvent from the main reactor. The precipitation reactor defines a precipitation zone, wherein the solution is treated to precipitate the 3-phosphoglycerate. In particular, the solution can be treated by introducing a precipitation reagent into the precipitation reactor to facilitate precipitation of the 3-phosphoglycerate. Accordingly, the precipitation reactor may optionally comprise a precipitation agent inlet for introducing the precipitation agent into the precipitation reactor.

In addition, the systems of the present disclosure optionally comprise a centrifuge operatively connected to the main reactor or, if used, the precipitation reactor. The centrifuge receives the precipitate comprising the 3-phosphoglycerate and the solvent. The centrifuge is operated to separate the precipitate from the solvent. Further, an optional recycle stream can be provided for receiving the solvent from the centrifuge and for supplying the solvent to the main reactor. Alternatively, some or all of the recycled solvent can be diverted for one or more other uses. In addition, a dryer can be optionally provided for receiving and drying the 3-phosphoglycerate from the centrifuge. The dryer can use any of a variety of techniques for drying the 3-phosphoglycerate including, but not being limited to, air drying, oven drying, spray drying and combinations thereof.

The present disclosure further provides the following embodiments:

1. A method of sequestering carbon dioxide comprising: reacting carbon dioxide with an organic substrate, a carboxylase, and a salt under conditions suitable to form a large scale amount of 3-phosphoglycerate.

2. The method of clause 1, wherein the organic substrate is a D-ribulose 1,5-bisphosphate sodium salt hydrate.

3. The method of any one of clauses 1 and 2, wherein the carboxylase is a D-ribulose 1,5-diphosphate carboxylase.

4. The method of any one of clauses 1-3, wherein the salt is magnesium chloride.

5. The method of any one of clauses 1-4, wherein the reacting step is performed in a solution.

6. The method of clauses 5, wherein the solution comprises a buffer.

7. The method of any one of clauses 5 and 6 wherein the solution comprises an anti-foaming agent.

8. The method of any one of clauses 1-7, further comprising separating the carbon dioxide from a mixture of gases.

9. The method of any one of clauses 1-8, further comprising obtaining the carboxylase from spinach.

10. The method of any one of clauses 1-9, further comprising forming the ribulose 1,5-diphosphate carboxylase by a batch process.

11. The method of any one of clauses 1-9, further comprising forming the ribulose 1,5-diphosphate carboxylase by a flow process.

12. The method of any one of clauses 1-11, wherein the reacting step comprises reacting carbon dioxide with the organic substrate, the carboxylase, and the salt at a temperature of about 15 to about 50° C.

13. The method of any one of clauses 1-12, wherein the reacting step comprises reacting carbon dioxide with the organic substrate, the carboxylase, and the salt for about 1 to about 6 hours.

14. The method of any one of clauses 1-13, wherein the reacting step comprises reacting carbon dioxide with the organic substrate, the carboxylase, and the salt at a pH of about 6 to about 8.

15. The method of any one of clauses 1-14, further comprising the isolating the 3-phosphoglycerate.

16. The method of clause 15, wherein the isolating the 3-phosphoglycerate comprises separating the 3-phosphoglycerate from a solvent.

17. The method of clause 16, wherein the isolating the 3-phosphoglycerate comprises recycling the solvent.

18. The method of any one of clauses 16 and 17, wherein the solvent is water.

19. The method of any one of clauses 15-18, wherein the isolating the 3-phosphoglycerate comprises precipitating the 3-phosphoglycerate.

20. The method of any one of clauses 15-19, wherein the isolating the 3-phosphoglycerate comprises drying the 3-phosphoglycerate.

21. The method of clause 20, wherein the drying the 3-phosphoglycerate comprises spray drying the 3-phosphoglycerate.

22. A process for forming a 3-phosphoglycerate comprising: reacting carbon dioxide with an organic substrate, a carboxylase, and a salt under conditions suitable to form a large scale amount of the 3-phosphoglycerate.

23. A system for sequestering carbon dioxide comprising a main reactor, wherein the main reactor comprises: a carbon dioxide inlet for introducing carbon dioxide into the main reactor; and a 3-phosphoglycerate outlet for removing 3-phosphoglycerate from the main reactor, wherein the main reactor defines a reaction zone for reacting the carbon dioxide with an organic substrate, a carboxylase and a salt.

24. The system of clause 23, wherein the reaction zone is at least 100,000 L.

25. The system of any one of clauses 23 and 24, further comprising a carboxylase inlet for introducing a carboxylase into the main reactor.

26. The system of any one of clauses 23-25, further comprising a flow reactor operatively connected to the carboxylase inlet for continuously flowing the carboxylase into the main reactor.

27. The system of any one of clauses 23-26, further comprising an anti-foaming agent inlet for introducing an anti-foaming agent into the main reactor.

28. The system of any one of clauses 23-27, further comprising a precipitation reactor operatively connected to the 3-phosphoglycerate outlet for receiving a solution comprising the 3-phosphoglycerate and a solvent from the main reactor, wherein the precipitation reactor defines a precipitation zone for reacting the solution with a precipitation agent to form a precipitate comprising the 3-phosphoglycerate.

29. The system of clause 28, wherein the precipitation reactor comprises a precipitation agent inlet for introducing the precipitation agent into the precipitation reactor.

30. The system of any one of clauses 23-29, further comprising a centrifuge operatively connected to the precipitation reactor for receiving the 3-phosphoglycerate and the solvent and for separating the 3-phosphoglycerate from the solvent.

31. The system of clause 30, further comprising a recycle stream operatively connected to the centrifuge for receiving the solvent and operatively connected to the main reactor for supplying the solvent to the main reactor.

32. The system of any one of clauses 30 and 31, further comprising a dryer operatively connected to the centrifuge for receiving and drying the 3-phosphoglycerate.

### EXAMPLES

The following examples are merely illustrative, and do not limit this disclosure in any way.

Reaction Scheme to make 3-Phosphoglycerate:

The reaction scheme used for making 3-phosphoglycerate in the following examples was:

RuDP+CO₂+RUBISCO in Trizma+MgCl₂→2 moles of 3-Phosphoglycerate

wherein:
RuDP: D-Ribulose 1,5-Bisphosphate Sodium Salt Hydrate;
RUBISCO: D-Ribulose 1,5-diphosphate Carboxylase from Spinach;
CO_{2:} Carbon dioxide;
MgCl₂: Magnesium chloride (0.1 M); and
Trizma base 1 M solution.

### Example 1

Reaction of Carbon Dioxide with 50 mg D-Ribulose 1,5-Bisphosphate Sodium Salt Hydrate, D-Ribulose 1,5-Diphosphate Carboxylase and Magnesium Chloride

A 50 mg aliquot of D-Ribulose 1,5-bisphosphate (RuDP) was placed in a 50 ml three neck round bottom flask, dissolved in 6.5 ml of de-ionized water, and mixed with a magnetic stir bar. The air in the flask was replaced with CO₂ on the surface of the solution for 5 minutes and purged for 3 minutes to remove oxygen/air from the solution.

1×1 UN of D-Ribulose 1,5-diphosphate carboxylase from Spinach (RUBISCO) was dissolved in 1 ml of Trizma buffer (1 M). A 1 ml portion of the resulting RUBISCO solution was added to the flask and the reaction mixture was stirred.

A 0.32 ml aliquot of magnesium chloride (0.1 M) was added to the flask and the reaction mixture was stirred.

Carbon dioxide gas was bubbled through the reaction mixture for 6 hours with the addition being monitored through a gas bubbler. The rate of CO₂ addition was adjusted to avoid overflow from foaming of the reaction mixture. After 6 hours, the CO₂ addition was stopped and the reaction mixture was lyophilized.

The reaction mixture was tested by ¹H NMR and the formation of 3-phosphoglycerate was confirmed.

### Examples 2-4

Reaction of Carbon Dioxide with 500 mg D-Ribulose 1,5-Bisphosphate Sodium Salt Hydrate, D-Ribulose 1,5-Diphosphate Carboxylase and Magnesium Chloride

The following procedure was repeated three times (Examples 2-4) to verify the reproducibility and consistency of 3-phosphoglycerate formation. For each of Examples 2-4, a 500 mg aliquot of D-Ribulose 1,5-bisphosphate (RuDP) was placed in a 500 ml three neck round bottom flask, dissolved in 65 ml of de-ionized water, and mixed. The air in the flask was replaced with CO₂ on the surface of the solution for 5 minutes and purged for 3 minutes to remove oxygen/air from the solution.

2×5 UN of D-Ribulose 1,5-diphosphate carboxylase from Spinach (RUBISCO) was dissolved in 2×5 ml of Trizma buffer (1M). A 10 ml portion of the resulting RUBISCO solution was added to the flask and the reaction mixture was stirred.

A 3.2 ml aliquot of magnesium chloride (0.1M) was added to the flask and the reaction mixture was stirred.

Carbon dioxide gas was bubbled through the reaction mixture for 6 hours with the addition being monitored through a gas bubbler. The rate of CO₂ addition was adjusted to avoid overflow from foaming of the reaction mixture. After 6 hours, the CO₂ addition was stopped and the reaction mixture was lyophilized.

The reaction mixture was tested by ¹H, ¹³C and ³¹P NMRs and the formation of 3-phosphoglycerate was confirmed.

### Example 5

Reaction of ¹³ C labelled Carbon Dioxide with 500 mg D-Ribulose 1,5-Bisphosphate Sodium Salt Hydrate, D-Ribulose 1,5-Diphosphate Carboxylase and Magnesium Chloride

The 500 mg scale experiments (Examples 2-4) were repeated using ¹³C labelled carbon dioxide. ¹³C NMR and mass spectroscopy were used to prove the formation of 3-phosphoglycerate.

### Example 6

Reaction of Carbon Dioxide with 500 mg D-Ribulose 1,5-Bisphosphate Sodium Salt Hydrate, D-Ribulose 1,5-Diphosphate Carboxylase and Magnesium Chloride with Anti-Foaming Agent

The procedure of Examples 2-4 was repeated except that an anti-foaming agent was added to the reaction mixture. In particular, a 500 mg aliquot of D-Ribulose 1,5-bisphosphate (RuDP) was placed in a 500 ml three neck round bottom flask, dissolved in 65 ml of de-ionized water, and mixed. The air in the flask was replaced with CO₂ on the surface of the solution for 5 minutes and purged for 3 minutes to remove oxygen/air from the solution.

2×5 UN of D-Ribulose 1,5-diphosphate carboxylase from Spinach (RUBISCO) was dissolved in 2×5 ml of Trizma buffer (1M). A 10 ml portion of the resulting RUBISCO solution was added to the flask and the reaction mixture was stirred.

A 3.2 ml aliquot of magnesium chloride (0.1M) was added to the flask and the reaction mixture was stirred.

The anti-foaming agent was prepared by diluting Antifoam B Emulsion (Sigma-Aldrich, St. Louis, Mo., USA) in a 1:10 (vol) ratio with cold water. After the addition of magnesium chloride, a 1 ml aliquot of the prepared anti-foaming agent was added to the reaction mixture. The reaction mixture was then heated to 39° C. in a water bath.

Carbon dioxide gas was bubbled through the reaction mixture for 2 hours with the addition being monitored through a gas bubbler. After 2 hours, the CO₂ addition was stopped and the reaction mixture was lyophilized.

The reaction mixture was tested by ¹H NMR and the formation of 3-phosphoglycerate was confirmed.

All patents and publications cited herein are incorporated by reference into this application in their entirety and for all purposes as if fully set forth herein.

What is claimed is:

1. A method of sequestering carbon dioxide comprising:
reacting carbon dioxide with an organic substrate, a carboxylase, and a salt under conditions suitable to form a large scale amount of 3-phosphoglycerate.

2. The method of claim 1, wherein the organic substrate is a D-ribulose 1,5-bisphosphate sodium salt hydrate.

3. The method of claim 1, wherein the carboxylase is a D-ribulose 1,5-diphosphate carboxylase.

4. The method of claim 1, wherein the salt is magnesium chloride.

5. The method of claim 1, wherein the reacting step is performed in a solution.

6. The method of claim 5, wherein the solution comprises a buffer.

7. The method of claim 5, wherein the solution comprises an anti-foaming agent.

8. The method of claim 1, further comprising separating the carbon dioxide from a mixture of gases.

9. The method of claim 1, further comprising obtaining the carboxylase from spinach.

10. The method of claim 1, further comprising forming the ribulose 1,5-diphosphate carboxylase by a batch process.

11. The method of claim 1, further comprising forming the ribulose 1,5-diphosphate carboxylase by a flow process.

12. The method of claim 1, wherein the reacting step comprises reacting carbon dioxide with the organic substrate, the carboxylase, and the salt at a temperature of about 15 to about 50° C.

13. The method of claim 1, wherein the reacting step comprises reacting carbon dioxide with the organic substrate, the carboxylase, and the salt for about 1 to about 6 hours.

14. The method of claim 1, wherein the reacting step comprises reacting carbon dioxide with the organic substrate, the carboxylase, and the salt at a pH of about 6 to about 8.

15. The method of claim 1, further comprising the isolating the 3-phosphoglycerate.

16. The method of claim 15, wherein the isolating the 3-phosphoglycerate comprises separating the 3-phosphoglycerate from a solvent.

17. The method of claim 16, wherein the isolating the 3-phosphoglycerate comprises recycling the solvent.

18. The method of claim 16, wherein the solvent is water.

19. The method of claim 15, wherein the isolating the 3-phosphoglycerate comprises precipitating the 3-phosphoglycerate.

20. The method of claim 15, wherein the isolating the 3-phosphoglycerate comprises drying the 3-phosphoglycerate.

21. The method of claim 20, wherein the drying the 3-phosphoglycerate comprises spray drying the 3-phosphoglycerate.

22. A process for forming a 3-phosphoglycerate comprising:
reacting carbon dioxide with an organic substrate, a carboxylase, and a salt under conditions suitable to form a large scale amount of the 3-phosphoglycerate.

23. A system for sequestering carbon dioxide comprising a main reactor, wherein the main reactor comprises:
a carbon dioxide inlet for introducing carbon dioxide into the main reactor; and
a 3-phosphoglycerate outlet for removing 3-phosphoglycerate from the main reactor,
wherein the main reactor defines a reaction zone for reacting the carbon dioxide with an organic substrate, a carboxylase and a salt.

24. The system of claim 23, wherein the reaction zone is at least 100,000 L.

25. The system of claim 23, further comprising a carboxylase inlet for introducing a carboxylase into the main reactor.

26. The system of claim 23, further comprising a flow reactor operatively connected to the carboxylase inlet for continuously flowing the carboxylase into the main reactor.

27. The system of claim 23, further comprising an anti-foaming agent inlet for introducing an anti-foaming agent into the main reactor.

28. The system of claim 23, further comprising a precipitation reactor operatively connected to the 3-phosphoglycerate outlet for receiving a solution comprising the 3-phosphoglycerate and a solvent from the main reactor, wherein the precipitation reactor defines a precipitation zone for reacting the solution with a precipitation agent to form a precipitate comprising the 3-phosphoglycerate.

29. The system of claim 28, wherein the precipitation reactor comprises a precipitation agent inlet for introducing the precipitation agent into the precipitation reactor.

30. The system of claim 23, further comprising a centrifuge operatively connected to the precipitation reactor for receiving the 3-phosphoglycerate and the solvent and for separating the 3-phosphoglycerate from the solvent.

31. The system of claim 30, further comprising a recycle stream operatively connected to the centrifuge for receiving the solvent and operatively connected to the main reactor for supplying the solvent to the main reactor.

32. The system of claim 30, further comprising a dryer operatively connected to the centrifuge for receiving and drying the 3-phosphoglycerate.
